(19) United States
(12) Patent Application Publication
(10) Pub. No.: US 2019/0239772 A1
(43) Pub. Date: Aug. 8, 2019

(54) DETECTING RESPIRATION RATE

(71) Applicant: Bose Corporation, Framingham, MA (US)

(72) Inventors: Victoria A. GRACE, Framingham, MA (US); Harsh A. MANKODI, Brighton, MA (US); Jack E. READ, Marlborough, MA (US)

(21) Appl. No.: 15/888,582

(22) Filed: Feb. 5, 2018

(51) Int. Cl. A61B 5/08; A61B 7/00; A61B 5/00; A61B 5/113; A61B 5/024; H04R 1/10; H04R 3/04

(52) U.S. Cl. CPC A61B 5/0816; A61B 7/003; A61B 5/725; A61B 5/113; H04R 1/1041; A61B 5/7257; A61B 5/02438; H04R 1/1016; H04R 3/04; A61B 5/6817

(57) ABSTRACT

Aspects of the present disclosure provide a system including an earphone and a processor. The earphone includes at least one of a microphone or accelerometer/gyroscope. The system is configured to determine a user's respiration rate (breath rate per minute) either directly by measuring the breath rate or indirectly based on the user's heartrate signal.

### BACKGROUND

Aspects of the present disclosure relates to a system for detecting a respiration rate. The system includes an earphone having at least one of a microphone or an accelerometer/gyroscope.

A user's respiration rate (or breath rate per minute, BrPM) can provide an indication of the user's stress level, medical condition, or state of discomfort. There exists a need for a tool that can comfortably and discretely determine a user's respiration rate.

### SUMMARY

All examples and features mentioned herein can be combined in any technically possible manner.

According to an aspect, a system is provided including an earphone and processor. The earphone comprises a microphone, a housing surrounding the microphone, and an ear tip surrounding the housing and configured to acoustically couple the microphone to an ear canal of a user of the earphone and to acoustically close the entrance to the user's ear canal. The processor is configured to receive an input audio signal from the microphone and process the input audio signal to determine a respiration rate of the user. The processor is configured to process the input audio signal by: downsampling the input audio signal to obtain a downsampled signal, applying a bandpass filter to the downsampled signal to obtain a bandpass signal between a first frequency and a second frequency, performing envelope processing on the bandpass signal to obtain an envelope of the bandpass signal, performing a thresholding operation on the envelope of the bandpass signal to obtain a gated signal; and, determining the respiration rate based, at least in part, on the gated signal.

In an aspect, the first frequency is 200 hertz and the second frequency is 500 hertz. In an example, performing the envelope processing comprises processing the bandpass signal with an attack of 100 milliseconds and a release of 2,000 milliseconds.

In an aspect, the at least one processor is configured to process the input audio signal by: applying an envelope following operation to the gated signal to obtain a smoothed gated signal, wherein determining the respiration rate based, at least in part, on the gated signal comprises determining the respiration rate based on the smoothed gated signal.

In an aspect, the bandpass signal is compressed between the first and second frequency.

In an aspect, the processor is integrated within the earphone. In an aspect, the input audio signal comprises a combination of one or more of a breath signal from the user, body noise from the user, or noise external from the user.

According to an aspect, a system including an earphone and a processor is provided. The earphone comprises at least one of an accelerometer or a gyroscope, a housing surrounding the at least one accelerometer or gyroscope, and an ear tip surrounding the housing and configured to acoustically couple the at least one accelerometer or gyroscope to an ear canal of a user of the earphone and to acoustically close the entrance to the user's ear canal. The processor is configured to receive an input signal from the at least one of the accelerometer or gyroscope and process the input signal to determine a respiration rate of the user.

In an aspect, the processor is configured to process the input signal by applying a bandpass filter to the input signal to obtain a bandpass signal between a first frequency and a second frequency. In an aspect, the first frequency and the second frequency are below 1 hertz. In an aspect, the processor is configured to process the input signal by: transforming the bandpass signal from a time domain signal to a frequency domain signal, detecting one or more highest peaks in the frequency domain signal, and determining the respiration rate based, at least in part, on the highest peaks.

In an aspect, the processor is configured to process the input signal by: transforming the bandpass signal from a time domain signal to a frequency domain signal, perform a smoothing operation on the frequency domain signal to obtain a smoothed signal, detecting one or more highest peaks in the smoothed signal, and determining the respiration rate based, at least in part, on the highest peaks.

In an aspect, the processor is integrated within the earphone.

According to an aspect, a system including an earphone and a processor is provided. The earphone comprising a sensor configured to measure a heartbeat signal of a user of the earphone, a housing surrounding the sensor, and an ear tip surrounding the housing and configured to acoustically couple the sensor to an ear canal of a user of the earphone and to acoustically close the entrance to the user's ear canal. The processor is configured to receive an input signal based, at least in part, on the heartbeat signal of the user, from the sensor, perform peak detection on a version of the input signal to obtain at least one of a time and amplitude associated with detected peaks of the user's heartbeat signal, and determine a respiration rate of the user based, at least in part, on the obtained at least one of time and amplitude associated with the detected peaks.

According to an aspect, the system includes a second earphone comprising a second sensor configured to measure the heartbeat signal of a user of the earphone, a second housing surrounding the second sensor, and a second ear tip surrounding the second housing and configured to acoustically couple the second sensor to a second ear canal of the user of the earphone and to acoustically close the entrance to the user's second ear canal. The input signal further comprises the heartbeat signal measured from the second earphone. According to an aspect, the processor is configured to process the input signal by applying timing offset to the heartbeat signal measured from the second earphone.

According to an aspect, the processor is configured to process the input signal by performing a matched filter with one of a single or averaged heartbeat waveform to obtain a filtered signal, wherein the version of the input signal comprises the filtered signal.

According to an aspect, the processor is configured to determine the respiration rate by computing a distance between detected peaks of the user's heartbeat signal to obtain a coarse signal based, at least in part, on the obtained time associated with detected peaks, performing a smoothing operation of the coarse signal to obtain a smooth signal, and performing a fast Fourier transform (FFT) function on the smooth signal to obtain the respiration rate of the user.

According to an aspect, the processor is configured to determine the respiration rate by computing a heart rate signal of the user, based, at least in part, on the obtained amplitude of the detected peaks, performing a smoothing operation of the heart rate signal to obtain a smooth signal, and performing a fast Fourier transform (FFT) function on the smooth signal to obtain the respiration rate of the user.

According to an aspect, the processor is integrated within the earphone.

### BRIEF DESCRIPTION OF THE DRAWINGS

FIG. 1 is an external view of an earphone.

FIG. 2 is an example cross-section of the earphone.

FIG. 3 is an example algorithm to directly determine a respiration rate using a microphone in the earphone.

FIG. 4 is an example input signal receives via the microphone.

FIG. 5 is an example of a bandpass signal, compressed bandpass signal, and envelope of the compressed bandpass signal.

FIG. 6 is an example of a gated signal from which the respiration rate is determined.

FIG. 7 is an example algorithm to directly determine a respiration rate using an accelerometer/gyroscope.

FIG. 8 illustrates an example of X, Y, and Z components of a signal obtained by an accelerometer.

FIG. 9 illustrates an example of the bandpass signal of the X, Y, and Z components of the input signal.

FIG. 10 illustrates an example of the FFT signal of each of the X, Y, and Z components of the bandpass signal.

FIG. 11 illustrates an example algorithm to indirectly determine a respiration rate using an accelerometer/gyroscope.

FIG. 12 illustrates an example of the signal recorded by the accelerometer from the left and right earphones.

FIG. 13 illustrates an example signal after applying an offset and filtering.

FIG. 14 illustrates an example of a combined signal after a clean-up step.

FIG. 15 illustrates the signal after FFT, wherein the peaks represent the BrPM.

### DETAILED DESCRIPTION

Aspects described herein provide an earphone configured to determine a user's respiration rate. The earphone determines a user's respiration rate either directly or indirectly. Direct determination, sensing, or measurement of the user's respiration rate is based on a direct breath measurement. Indirect determination, sensing, or measurement of the user's respiration rate is based on determining the user's heart beat signal and correlating different aspects of the determined heart beat signal with the user's respiration rate. Example aspects of the determined heart beat signal include heart rate variability (HRV) and R-R peak intervals (RRi). In an aspect, the earphone described herein operates autonomously to determine the user's respiration rate. In another aspect, the earphone may communicate with an external processor, wherein the external processor uses the information obtained by the earphone to calculate the user's respiration rate.

The earphone configured to detect the respiration rate is small and comfortable for a user to wear while sleeping. The detected respiration rate may be used to any number of sleep staging, sleep entrainment, stress management, or blood pressure management applications. For example, to assist the user with falling to sleep, the earphone plays sounds that have a rhythm slightly slower than the user's own respiration rate. This naturally leads the user to slow their breathing to match the rhythm of the sounds, in a process referred to as entrainment. As the user slows their rate of respiration, the rate of the sounds is further reduced, in a feedback loop that leads the user gradually to sleep. Once the user falls asleep (as indicated by artifacts in their respiration rate), the earphone switches to playing masking sounds, which diminish the user's ability to detect, and be disturbed by, external sounds. If the user is detected to be waking up too early, entrainment may be reactivated. When it is time for the user to wake up, the system may coordinate wake-up sounds with the user's sleep state and other information to wake the user in the least-disruptive way possible. In other aspects the determined respiration rate is used to help calm a user down and lower their blood pressure.

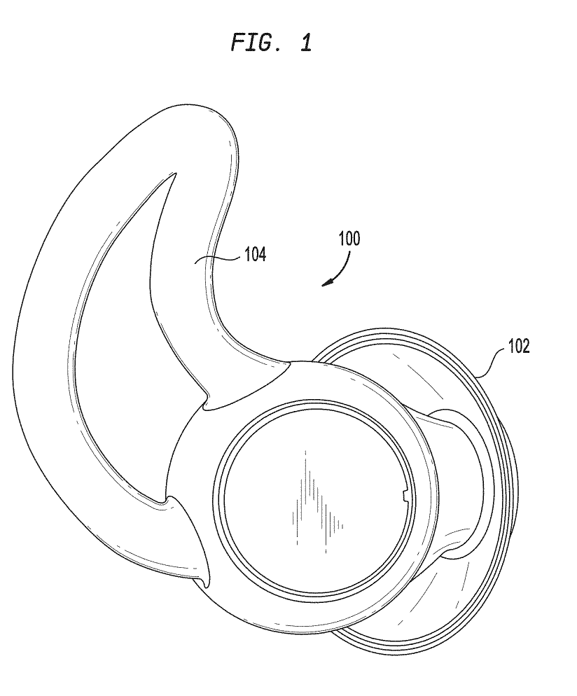

FIG. 1 illustrates an earphone 100 configured to detect a user's respiration rate. The earphone 100 includes an ear tip sealing structure 102 that blocks or occludes the entrance to the user's ear canal. A retaining structure 104 helps retain the earphone 100 in the user's ear. The retaining structure 104 provides pressure on the sealing structure 102 to maintain the seal by pushing on the concha, opposite to where the sealing structure meets the ear canal. The sealing structure 102 helps to passively block outside sounds from entering the ear. As a result of occluding the ear canal sounds produced by the body, such as the heartbeat and respiration sounds, are amplified within the ear canal.

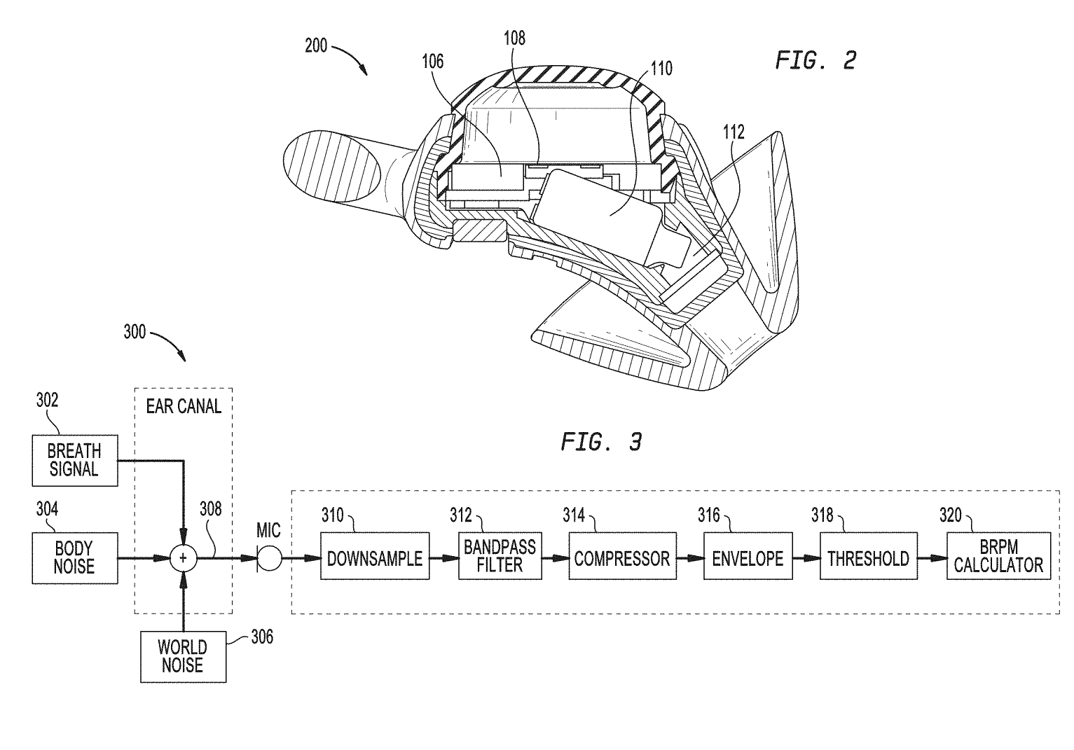

FIG. 2 illustrates a cross section 200 of the earphone 100. The earphone 100 includes a microphone 106 and an accelerometer/gyroscope 108; however, only one of a microphone, accelerometer, or gyroscope is necessary to determine the user's respiration rate as described herein. In an example, the earphone includes a balanced armature driver 110. The balanced armature driver 110 is not necessary to determine a respiration rate; however, it is used for enabling sleep staging, sleep entrainment, stress management, or blood pressure management applications. In an aspect, the earphone 100 includes a processor in area 112. The processor is configured to receive an input signal from the user of the earphone and process the signal to determine the user's respiration rate. The processor uses the determined respiration rate to adjust the timing of entrainment sounds being played to the user through a speaker or manage a user's blood pressure or stress level. In an example, the processor is integrated into the speaker housing. While the earphone includes a processor configured to calculate the user's respiration rate, in an aspect, the calculations may be performed by a processor external to the earphone, in a portable computing device.

### Directly Measuring a Respiration Rate Using a Microphone

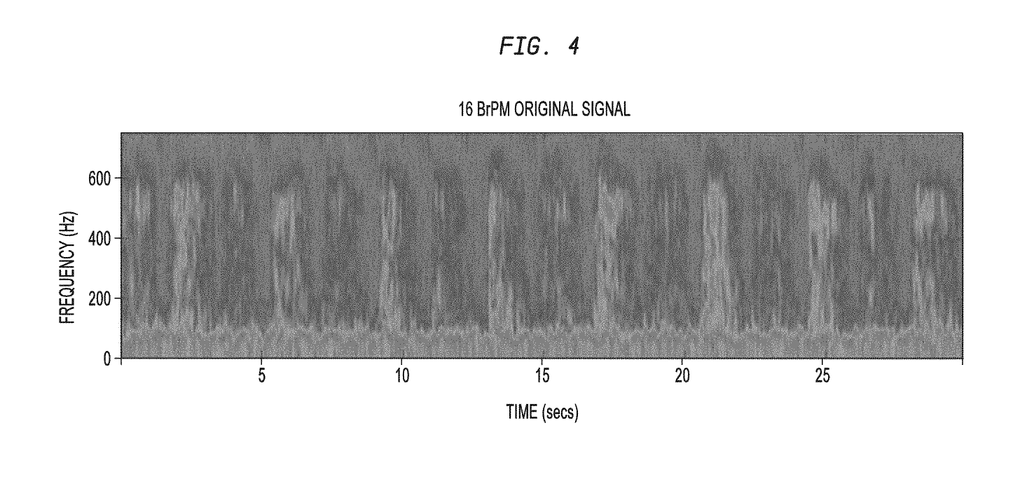

FIG. 3 illustrates an algorithm 300 for the earphone directly detecting the user's respiration rate using a microphone 106. The earphone 100 receives an input signal 308 associated with the user from the microphone 106. The input signal 308 includes any combination of the user's breath signal 302, the user's body noise 304, and noise external to the user 306. An example of an input signal is shown in FIG. 4.

The input signal is received at a higher sampling rate than necessary to determine the respiration rate. Therefore, at 310, the input signal is downsampled to obtain a downsampled signal. In an example, downsampling the input signal is performed by downsampling with anti-aliasing filter to a sampling rate of 1500 Hz.

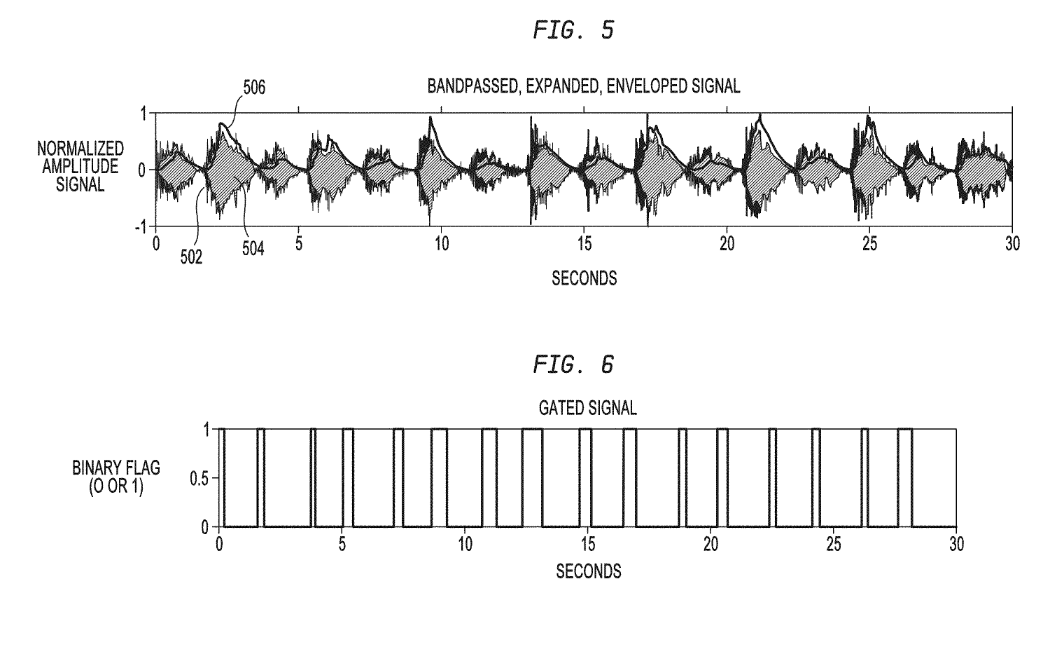

At 312, a bandpass filter is applied to the downsampled signal to obtain a bandpass signal between a first frequency and a second frequency. Filtering is performed to obtain a signal where a user's breath signal is most likely present. In an example, the first frequency is approximately 200 hertz and the second frequency is approximately 500 hertz. An example of a bandpass signal is illustrated at 502 in FIG. 5.

Optionally, at 314, the bandpass signal is compressed by a compressor/expander, to quiet the signal and make the pause between breathes more dramatic. As a result of this step, the bandpass signal is prepared for envelope following (316). An example of an expanded signal is illustrated at 504 in FIG. 5.

At 316, envelope processing is performed on either the bandpass signal obtained after bandpass filtering at 312 or the compressed bandpass signal obtained from the compressor/expander at 314 to obtain an envelope of the bandpass signal. The envelope processing is an envelope following operation wherein the absolute value of the signal is contoured. As an example, a leaky integrator is used to contour the absolute value of the signal. The attack and release time can be tuned to an attack of 100 milliseconds and a release of 2,000 milliseconds. According to an example, an adaptive filter is used, where how quickly the signal is changing determines the attack and release times. An example of the envelope of the bandpass signal is illustrated at 506 in FIG. 5.

At 318, a thresholding operation is performed on the envelope of the bandpass signal. The thresholding operation determines the pauses between a user's inhaling and exhaling to produce a gated signal. For example, having determined the outlined shape of the breath (as illustrated in 506 in FIG. 5), a threshold value is set. Anything below that threshold is assigned a one and anything above the threshold is assigned a zero. In an example, the threshold value is determined based on an initial calibration, where sound is recorded when the user is not breathing. As a result of the thresholding operation, an outline of the breath signal being on or off is produced, as shown in FIG. 6.

At 320, a respiration rate, or Breath Rate Per Minute (BrPM) is calculated based, at least in part on the gated signal. The gated signal is enveloped again to smooth out flutters of the gated signal which may appear if the enveloped signal amplitude fluctuate around the threshold. Once the first gated signal has been smoothed, a second threshold, set at the mean signal height, is used to create a new gated signal. From this gated signal, the breath rate is computed in one of two ways. In the time domain, the number of times the gated signal is on (turns to one) within 30 seconds is counted. This number determines the number of breaths per minute. In the frequency domain, an FFT of the gated signal is taken and the highest peak is the breath rate.

### Directly Measuring a Respiration Rate Using an Accelerometer or Gyroscope

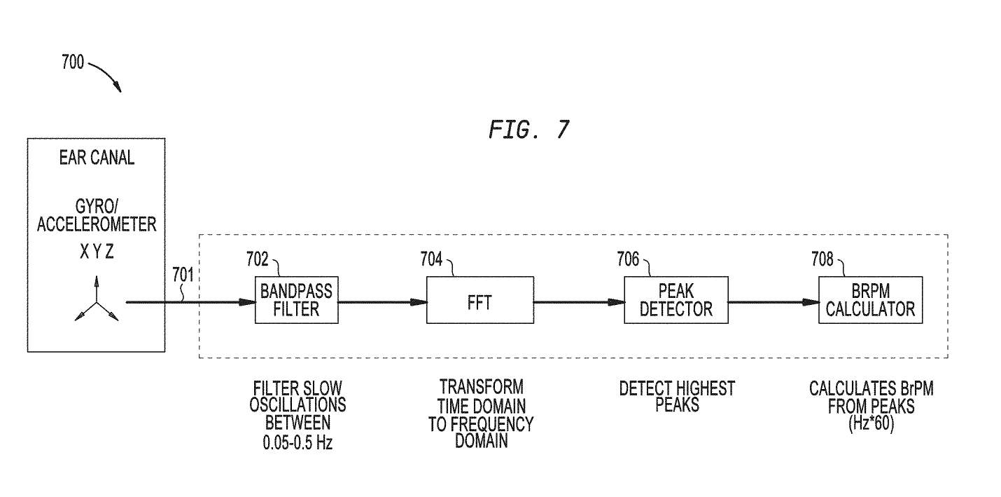

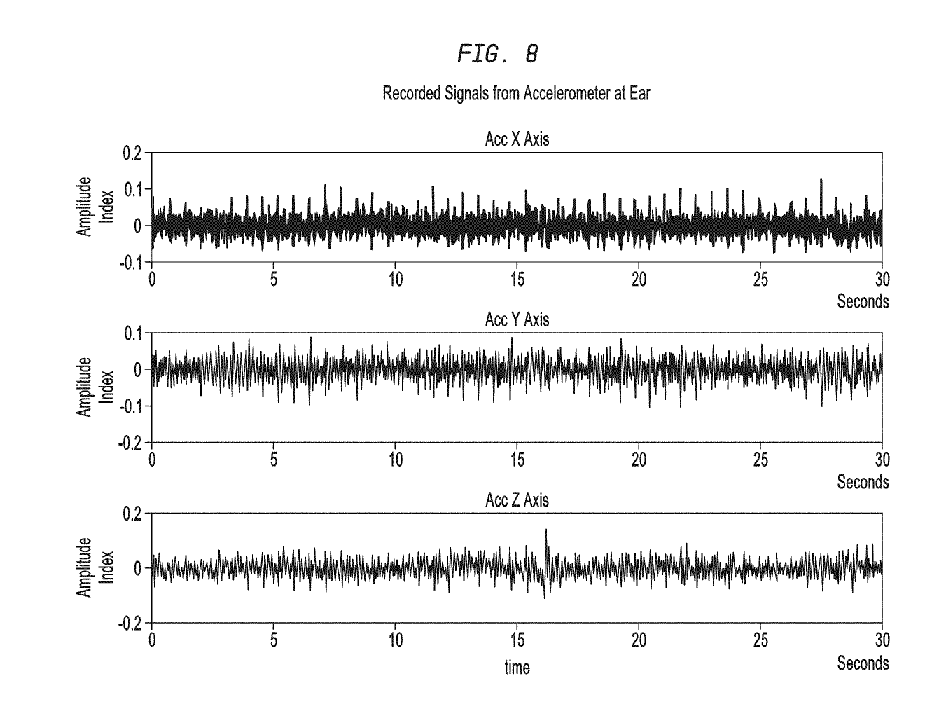

FIG. 7 illustrates an algorithm 700 for the earphone directly detecting the user's respiration rate using an accelerometer/gyroscope 108. An input signal 701 is acquired by the accelerometer or gyroscope. An example of X, Y, and Z components of a signal obtained by an accelerometer is illustrated in FIG. 8. According to an example, the signal is obtained from an earphone in one of the left or right ear of the user.

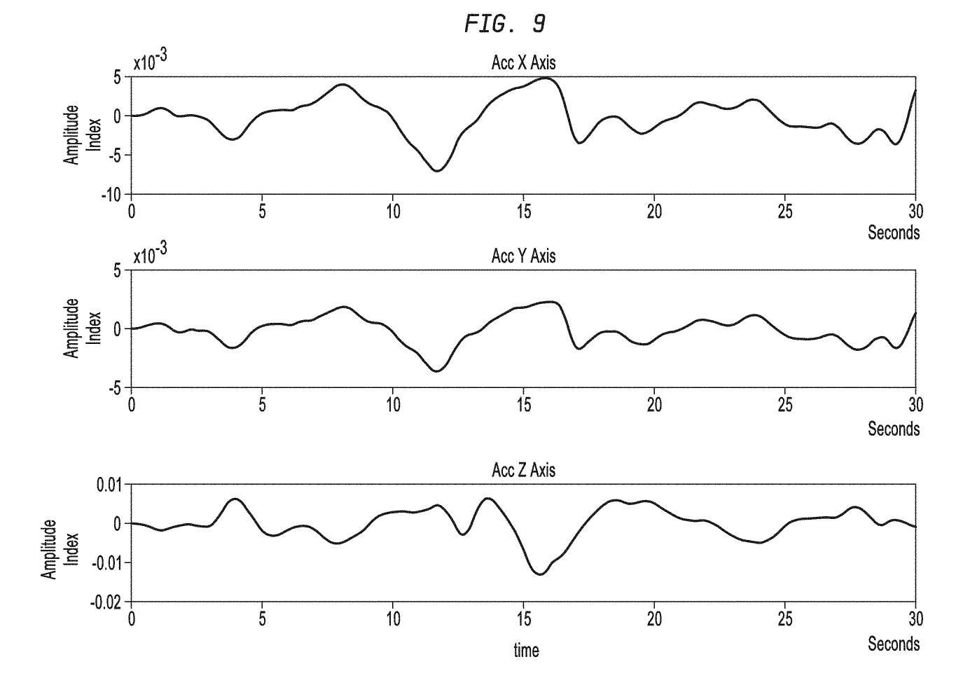

At 702, a bandpass filter is applied to the input signal to obtain a bandpass signal between a first frequency and a second frequency. In one example, the bandpass signal is between 0.05 hertz and 0.5 hertz. FIG. 9 illustrates an example of the bandpass signal of the X, Y, and Z components of the input signal. A high and low pass filter may be used instead of a single band pass.

After the bandpass filter is applied an optional smoothing operation, not illustrated in FIG. 7, is performed to smooth out the bandpass signal.

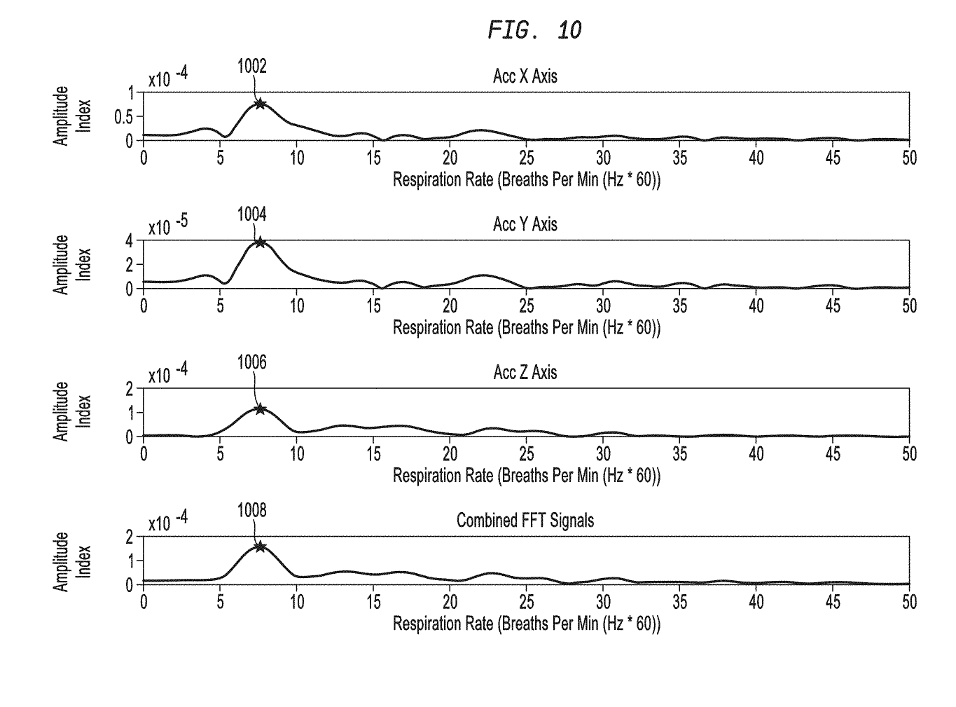

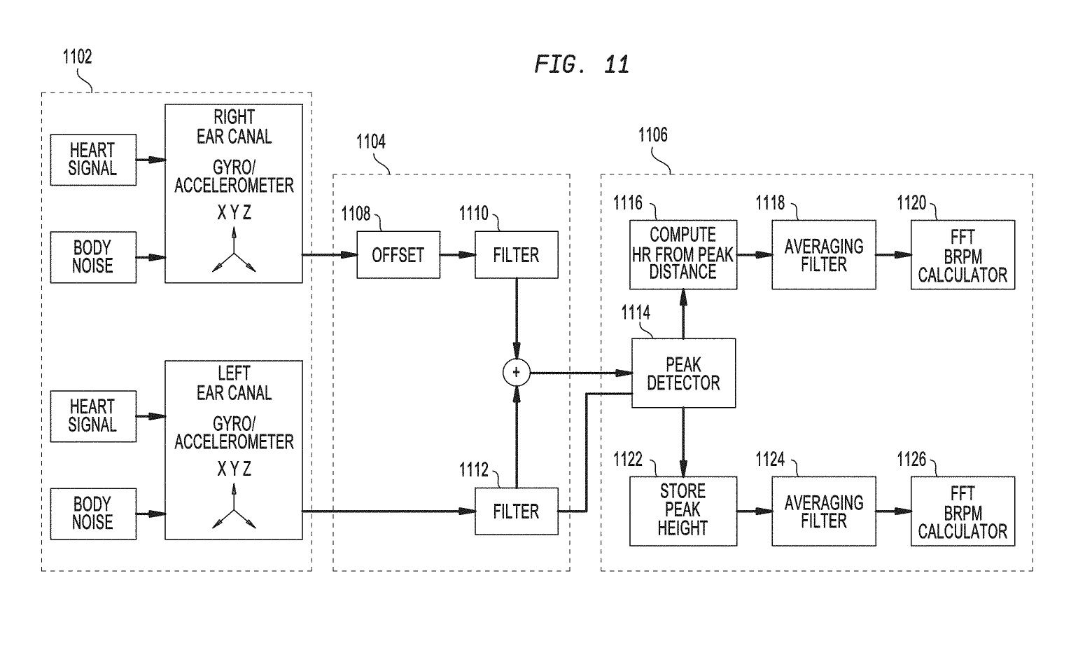

At 704, a Fast Fourier transform (FFT) operation is performed on either the bandpass signal or the smoothed bandpass signal to transform the signal from the time domain to frequency domain. FIG. 10 illustrates an example of the FFT signal of each of the X, Y, and Z components of the bandpass signal. FIG. 10 also illustrates an example of a combined FFT signal, resulting from combining the X, Y, and Z components of the FFT signal shown in FIG. 10.

At 706, one or more peaks (highest peaks) in the FFT signal are detected. According to an example, the highest peaks are detected in each of the X, Y, and Z components, as shown at 1002, 1004, and 1006. Additionally, or alternatively, the highest peak is detected in the combined signal, as shown at 1008.

At 708, the BrPM is calculated based on the detected peaks. The highest peak found within the range of expected breathing rate (corresponding to the filter cutoffs between 0.05 hertz and 0.5 hertz), determines the breath rate. Determined peak location in Hz multiplied by 60 equals the breath rate in breaths per minute. An optional smoothing process step may be added before this calculation to assist with accuracy performance.

### Indirectly Measuring a Respiration Rate

A microphone, accelerometer/gyroscope, or optical photoplethysmographer (PPG) sensor is used to indirectly determine a respiration rate as described herein. Specifically, a sensor is used to detect a heart beat signal. The user's respiration rate is determined based on the detected heart beat signal.

FIG. 11 illustrates an example algorithm for indirectly determining a user's respiration rate using an accelerometer/gyroscope. The algorithm in FIG. 11 is divided into three portions: acquisition 1102, clean-up 1104, and heart beats to BrPM calculation 1106.

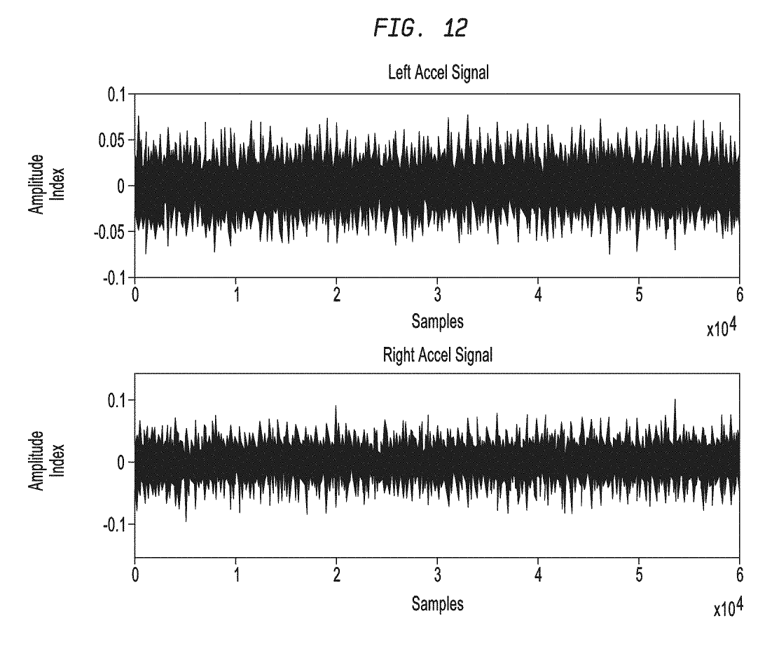

During the acquisition portion 1102 of the algorithm, the heartbeat signal is recorded by an accelerometer 108 on the earphone at least one of the user's ear. The accelerometer records the heartbeat signal as well and other body noise. In one example, the heartbeat signal is recorded using a sensor on each of the earphones; however, the user's respiration rate can be determined using a heartbeat signal recorded from one earphone. FIG. 12 illustrates an example of the signal recorded by the accelerometer from the left and right earphones. While an accelerometer is provided as an example, a gyroscope, microphone, or other sensor can be used to record the user's heartbeat signal.

During the clean-up step 1104 of the algorithm, the recorded in signal is cleaned-up in an effort to more clearly define, and make more obvious, the heartbeat peaks. Optionally, at 1108, an offset is applied to the signal recorded in by the right earphone. The offset attempts to correct the slight time-offset of between the peaks obtained from the right and the left earphones.

Once the signals recorded in by the right and left earphones are time-aligned, the signals are filtered at 1110, 1112. One of a bandpass filter, low pass filter, high pass filter, wavelet de-noising using soft or hard thresholding, or matched filtering may be applied to the signals at 1110, 1112. The filtered right and left signals are combined during the clean-up step. Before combining, the absolute value of the signals is taken to account for potential flipped phase between the left and right signals. While FIG. 11 illustrates adding the signals, the signals could also be multiplied. 1402 shows the absolute value signals combined.

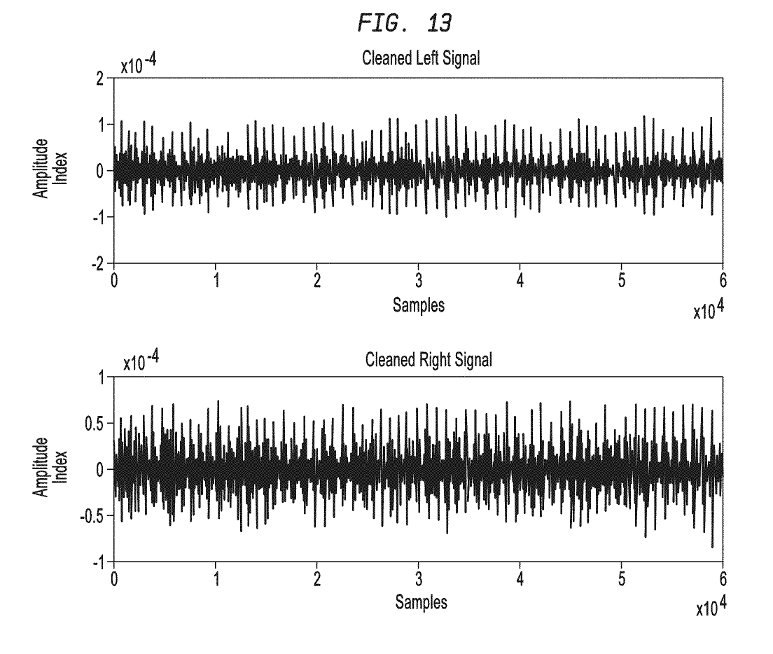

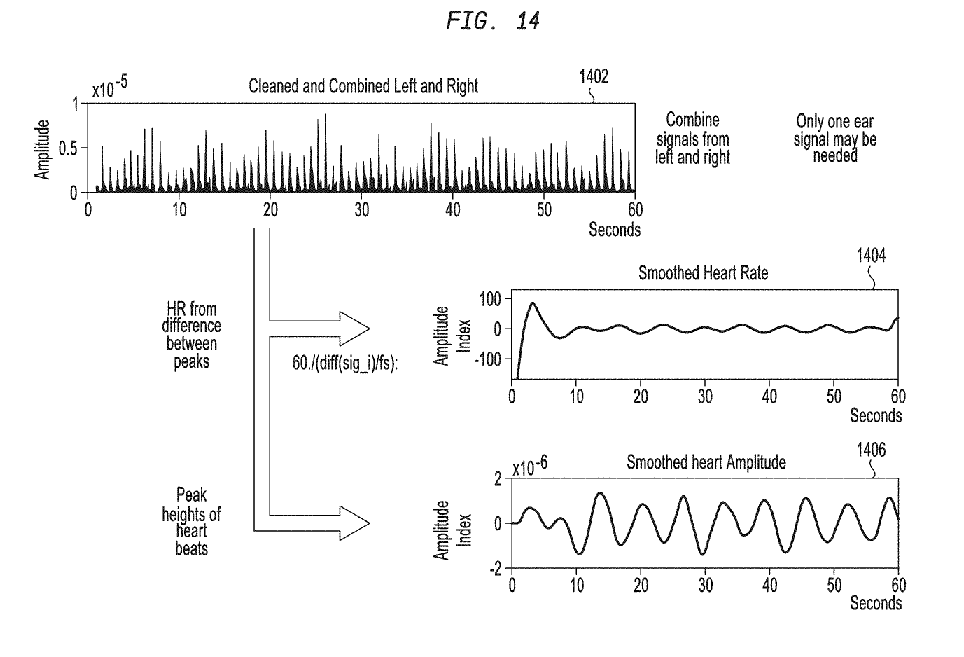

FIG. 13 illustrates an example of the signals after the offset is applied to the signal recorded in by the right earphone and the signals have been filtered. The signal recorded in from the right earphone has been shifted by a calculated offset. The shifted signal and the signal recorded in from the left earphone were filtered using a matched filter with a single or averaged heartbeat waveform to produce more defined (more visible) heartbeat signals shown in FIG. 13.

FIG. 14 illustrates an example of the combined signal at 1402, after the clean-up step 1104. This is the absolute value of the signal post-matched filtering, which helps combine the signals without destructive interference. As noted above, the signal may not need to be combined if a recorded in signal is used from only one earphone.

At 1114, the heartbeat peaks are detected from the signal obtained as a result of the clean-up step 1104. After peak detection, one of two methods is used to determine the BrPM using this signal. According to a first method, the BrPM is determined based on a distance between detected peaks. According to a second method, the BrPM is determined based on the amplitude (height) of the detected peaks.

At 1116, the distances between peaks in the clean-up signal is obtained. Signal I is a vector storing the time locations (in milliseconds) where each of the peaks occurs. The difference is calculated from peak-to-peak (from neighboring numbers in vector I). The result is divided by the sampling rate fs). Thereafter, point wise division is performed, where 60 is divided by each of these values. This result may be smoothed, at 1118, to obtain a smoothed heart rate as signal shown at 1404 in FIG. 14; however smoothing is not necessary. The frequency of the oscillating wave form shown at 1404 is the breath rate.

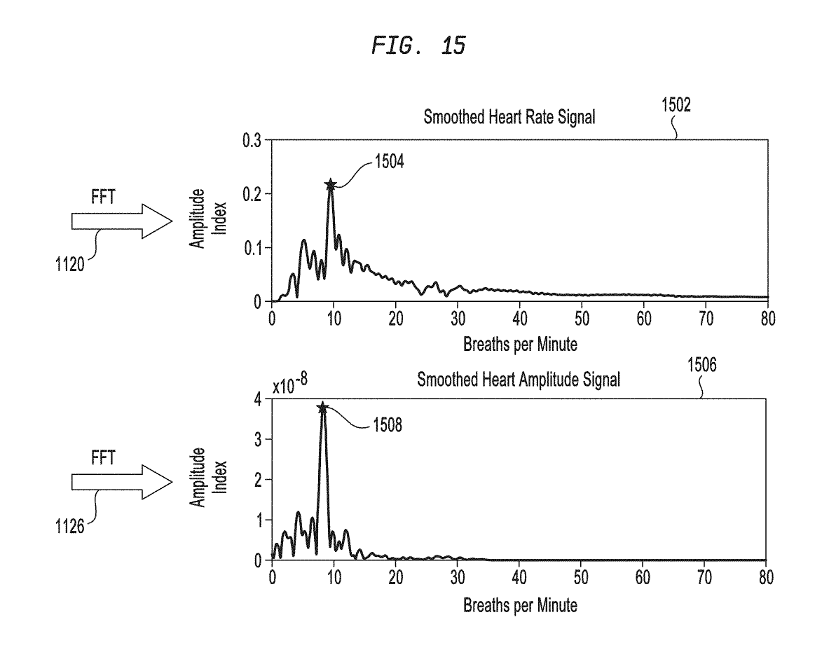

At 1120, an FFT is performed on the signal 1404 to produce the signal 1502 in FIG. 15. The highest peak 1504 is the BrPM.

According to the second method, at 1122, the heartrate peak heights are stored. A contour of the signal 1402 is determined at 1124 by taking the heights of the peaks and smoothing with a moving average filter. The smoothed heartrate amplitude signal 1406 is obtained. At 1126, an FFT is performed on the signal 1406 to produce the signal 1506 in FIG. 15. The highest peak 1508 is the BrPM.

While the indirect method of determining a user's BrPM was shown with a specific example to signals obtained using an accelerometer, any sensor that records a heartbeat signal can be used to determine the user's BrPM using the steps described in the heart beats to BrPM calculation 1106 portion of the algorithm 1100.

Several of the above-referenced applications describe a system that detects a user's respiration rate. The respiration rate may be determined either directly or indirectly using a sensor on an earphone. The determined respiration rate may be used by the earphone or other devices to help manage a user's sleep, stress, or health.

Embodiments of the systems and methods described above comprise computer components and computer-implemented steps that will be apparent to those skilled in the art. For example, it should be understood by one of skill in the art that the computer-implemented steps may be stored as computer-executable instructions on a computer-readable medium such as, for example, hard disks, optical disks, solid-state disks, flash ROMS, nonvolatile ROM, and RAM. Furthermore, it should be understood by one of skill in the art that the computer-executable instructions may be executed on a variety of processors such as, for example, microprocessors, digital signal processors, and gate arrays. For ease of exposition, not every step or element of the systems and methods described above is described herein as part of a computer system, but those skilled in the art will recognize that each step or element may have a corresponding computer system or software component. Such computer system and software components are therefore enabled by describing their corresponding steps or elements (that is, their functionality), and are within the scope of the disclosure.

A number of implementations have been described. Nevertheless, it will be understood that additional modifications may be made without departing from the scope of the inventive concepts described herein, and, accordingly, other embodiments are within the scope of the following claims.

The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but is to be accorded the widest scope consistent with the principles and novel features disclosed herein.

1. A system comprising:
an earphone comprising: a microphone, a housing surrounding the microphone, and an ear tip surrounding the housing and configured to acoustically couple the microphone to an ear canal of a user of the earphone and to acoustically close the entrance to the user's ear canal; and
a processor configured to:
receive an input audio signal from the microphone; and
process the input audio signal to determine a respiration rate of the user, wherein the processor is configured to process the input audio signal by:
downsampling the input audio signal to obtain a downsampled signal;
applying a bandpass filter to the downsampled signal to obtain a bandpass signal between a first frequency and a second frequency;
performing envelope processing on the bandpass signal to obtain an envelope of the bandpass signal;
performing a thresholding operation on the envelope of the bandpass signal to obtain a gated signal; and
determining the respiration rate based, at least in part, on the gated signal.

2. The system of claim 1, wherein the first frequency is 200 hertz and the second frequency is 500 hertz.

3. The system of claim 1, wherein performing the envelope processing comprises:
processing the bandpass signal with an attack of 100 milliseconds and a release of 2,000 milliseconds.

4. The system of claim 1, wherein the at least one processor is configured to process the input audio signal by:
applying an envelope following operation to the gated signal to obtain a smoothed gated signal,
wherein determining the respiration rate based, at least in part, on the gated signal comprises determining the respiration rate based on the smoothed gated signal.

5. The system of claim 1, wherein the bandpass signal is compressed between the first and second frequency.

6. The system of claim 1, wherein the processor is integrated within the earphone.

7. The system of claim 1, wherein the input audio signal comprises a combination of one or more of a breath signal from the user, body noise from the user, or noise external from the user.

8. A system comprising:
an earphone comprising: at least one of an accelerometer or a gyroscope, a housing surrounding the at least one accelerometer or gyroscope, and an ear tip surrounding the housing and configured to acoustically couple the at least one accelerometer or gyroscope to an ear canal of a user of the earphone and to acoustically close the entrance to the user's ear canal; and
a processor configured to:
receive an input signal from the at least one of the accelerometer or gyroscope; and
process the input signal to determine a respiration rate of the user.

9. The system of claim 8, wherein the processor is configured to process the input signal by:
applying a bandpass filter to the input signal to obtain a bandpass signal between a first frequency and a second frequency.

10. The system of claim 9, wherein the first frequency and the second frequency are below 1 hertz.

11. The system of claim 9, wherein the processor is configured to process the input signal by:
transforming the bandpass signal from a time domain signal to a frequency domain signal;
detecting one or more highest peaks in the frequency domain signal; and
determining the respiration rate based, at least in part, on the highest peaks.

12. The system of claim 9, wherein the processor is configured to process the input signal by:
transforming the bandpass signal from a time domain signal to a frequency domain signal;
perform a smoothing operation on the frequency domain signal to obtain a smoothed signal;
detecting one or more highest peaks in the smoothed signal; and
determining the respiration rate based, at least in part, on the highest peaks.

13. The system of claim 8, wherein the processor is integrated within the earphone.

14. A system comprising:
an earphone comprising a sensor configured to measure a heartbeat signal of a user of the earphone, a housing surrounding the sensor, and an ear tip surrounding the housing and configured to acoustically couple the sensor to an ear canal of a user of the earphone and to acoustically close the entrance to the user's ear canal; and
a processor configured to:
receive an input signal based, at least in part, on the heartbeat signal of the user, from the sensor;
perform peak detection on a version of the input signal to obtain at least one of a time and amplitude associated with detected peaks of the user's heartbeat signal; and
determine a respiration rate of the user based, at least in part, on the obtained at least one of time and amplitude associated with the detected peaks.

15. The system of claim 14, comprising:
a second earphone comprising a second sensor configured to measure the heartbeat signal of a user of the earphone, a second housing surrounding the second sensor, and a second ear tip surrounding the second housing and configured to acoustically couple the second sensor to a second ear canal of the user of the earphone and to acoustically close the entrance to the user's second ear canal,
wherein the input signal further comprises the heartbeat signal measured from the second earphone.

16. The system of claim 15, wherein the processor is configured to process the input signal by:
applying timing offset to the heartbeat signal measured from the second earphone.

17. The system of claim 14, wherein the processor is configured to process the input signal by:
performing a matched filter with one of a single or averaged heartbeat waveform to obtain a filtered signal, wherein the version of the input signal comprises the filtered signal.

18. The system of claim 14, wherein the processor is configured to determine the respiration rate by:
based, at least in part, on the obtained time associated with detected peaks, computing a distance between detected peaks of the user's heartbeat signal to obtain a coarse signal;
performing a smoothing operation of the coarse signal to obtain a smooth signal; and
performing a fast Fourier transform (FFT) function on the smooth signal to obtain the respiration rate of the user.

19. The system of claim 14, wherein the processor is configured to determine the respiration rate by:
based, at least in part, on the obtained amplitude of the detected peaks, computing a heart rate signal of the user;
performing a smoothing operation of the heart rate signal to obtain a smooth signal; and
performing a fast Fourier transform (FFT) function on the smooth signal to obtain the respiration rate of the user.

20. The system of claim 14, wherein the processor is integrated within the earphone.
